(19) United States
(12) Plant Patent Application Publication
(10) Pub. No.: US 2019/0357413 P1
(43) Pub. Date: Nov. 21, 2019

(54) Stokesia plant named 'Mini Mels'

(50) ;

(71) Applicant: Van Heesbeen Cultures B.V., Rijsbergen (NL)

(72) Inventor: Ad van Heesbeen, Rijsbergen (NL)

(73) Assignee: Van Heesbeen Cultures B.V., Rijsbergen (NL)

(21) Appl. No.: 16/501,668

(22) Filed: May 20, 2019

(30) Foreign Application Priority Data
May 18, 2018 (QZ) 2018/1336

(51) Int. Cl. A01H 6/14

(52) U.S. Cl. PLT/484

(57) ABSTRACT

A new and distinct Stokesia plant named ‘Mini Mels’ which is characterized by the combination of a compact globular habit, small dark purple flowers with yellow centers, and the stability of these characteristics from generation to generation.

### CROSS REFERENCE TO RELATED APPLICATIONS

This application claims priority to the Community Plant Variety Rights application number 2018/1336, filed May 18, 2018, which is herein incorporated by reference.

Latin name of the genus and species: The Latin name of the genus and species of the novel variety disclosed herein is Stokesia laevis.

Variety denomination: The inventive cultivar of Stokesia disclosed herein has been given the variety denomination ‘Mini Mels’.

### BACKGROUND OF THE INVENTION

Parentage: ‘Mini Mels’ is a seedling selection resulting from the controlled pollination of the seed parent, Stokesia laevis ‘Mels Blue’ (U.S. Plant Pat. No. 23,090), with an unnamed Stokesia laevis plant (not patented), the pollen parent. The crossing was made by the inventor in August of 2015 at a commercial greenhouse in Rijsbergen, the Netherlands. In August of 2016, one seedling from said cross was observed which exhibited smaller, dark purple flowers. The seedling was isolated for further evaluation in order to confirm the distinctness and stability of the characteristics first observed. Upon confirmation of distinctness and stability, ‘Mini Mels’ was selected for commercialization.

Asexual Reproduction: Asexual reproduction of ‘Mini Mels’, by way of softwood stem cuttings, was first performed in August of 2017 in Rijsbergen, the Netherlands. Through five subsequent generations, the unique features of this cultivar have proven to be stable and true to type.

### SUMMARY OF THE INVENTION

The cultivar ‘Mini Mels’ has not been observed under all possible environmental conditions. The phenotype may vary somewhat with variations in environment such as temperature, day length, and light intensity, without, however, any variance in genotype. The following traits have been repeatedly observed and are determined to be the unique characteristics of ‘Mini Mels’. These characteristics in combination distinguish ‘Mini Mels’ as a new and distinct Stokesia laevis cultivar:
1. Stokesia ‘Mini Mels’ exhibits a relatively compact plant size; and
2. Stokesia ‘Mini Mels’ exhibits a globular plant shape; and
3. Stokesia ‘Mini Mels’ exhibits small, dark purple flowers which are conspicuously centered yellow.

### BRIEF DESCRIPTION OF THE FIGURES

FIG. 1 illustrates, as nearly true as it is reasonably possible to make the same in color photographs of this type, an exemplary mature ‘Mini Mels’ plant grown outdoors at the inventor's commercial greenhouse in Rijsbergen, the Netherlands.

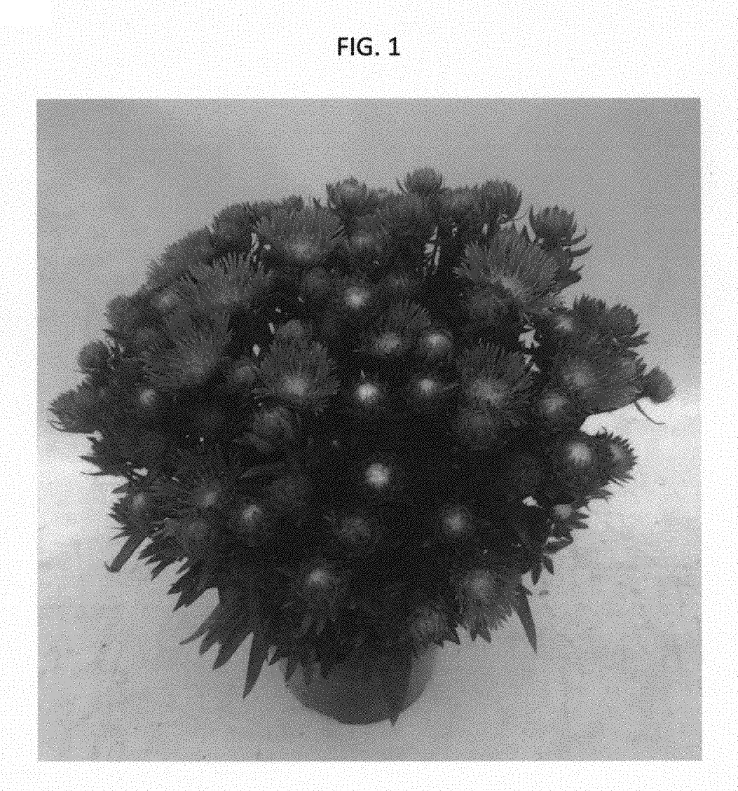

FIG. 2 illustrates, as nearly true as it is reasonably possible to make the same in color photographs of this type, the typical flowers of ‘Mini Mels’.

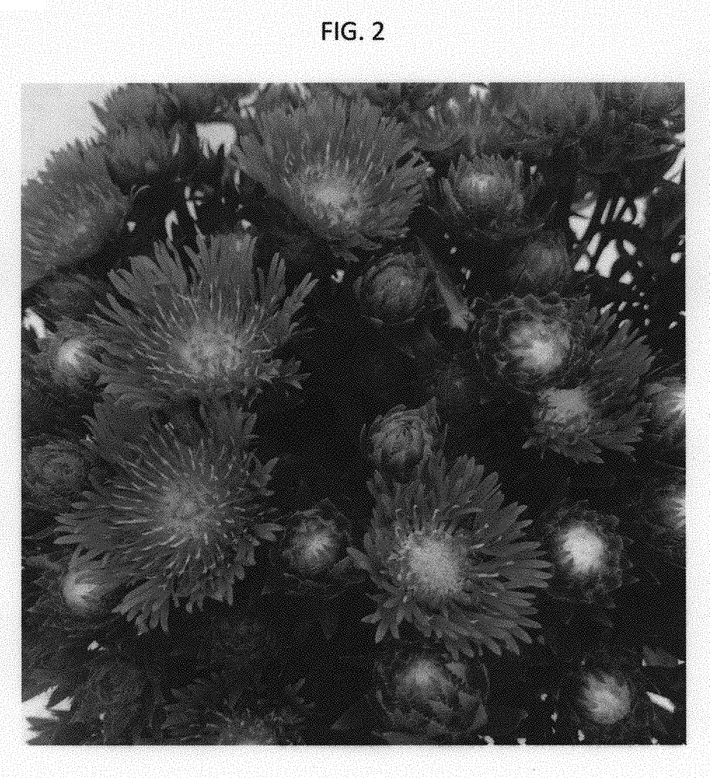

### BOTANICAL DESCRIPTION OF THE PLANT

A detailed botanical description of a new and distinct variety of Stokesia plant known as ‘Mini Mels’ is not yet available. A comparison with the parents and most similar variety of common knowledge are provided below.
General plant description: Botanical description is not yet available.
Root system: Botanical description is not yet available.
Stem: Botanical description is not yet available.
Foliage: Botanical description is not yet available.
Inflorescence: Botanical description is not yet available.
Flower buds: Botanical description is not yet available.
Flower: Botanical description is not yet available.
Reproductive organs: Botanical description is not yet available.
Seed and fruit: Botanical description is not yet available.

### COMPARISONS WITH THE PARENTS

Plants of the new cultivar ‘Mini Mels’ differ from its parent, Stokesia laevis ‘Mels Blue’ (U.S. Plant Pat. No. 23,090), by the characteristics described in Table 1.

TABLE 1

| Characteristic | ‘Mini Mels’ | ‘Mels Blue’ |
|---|---|---|
| Plant height. | Shorter than ‘Mels Blue’. | Taller than ‘Mini Mels’. |
| General coloration of | Dark purple and centered | Violet to violet-blue and |
| the flower. | yellow. | ceneterd white. |

Plants of the new cultivar ‘Mini Mels’ differ from its pollen parent, an unnamed Stokesia laevis plant (not patented), by the characteristics described in Table 2.

TABLE 2

| Characteristic | ‘Mini Mels’ | The pollen parent |
|---|---|---|
| Abundance of foliage. | More abundant. | Less abundant. |
| Foliage thickness. | Thicker than the pollen | Thinner than ‘Mini Mels’. |
| | parent. | |
| General coloration of | Darker green. | Lighter green. |
| mature foliage. | | |
| Floriferousness. | More flowers per plant. | Fewer flowers per plant. |
| General coloration of | Darker purple. | Lighter purple. |
| the flower. | | |

### COMPARISONS WITH THE MOST SIMILAR VARIETY OF COMMON KNOWLEDGE

Plants of the new cultivar ‘Mini Mels’ may be distinguished from the variety Stokesia laevis ‘Mega Mels’ (an application for which is being filed concurrently with the instant plant variety) by the combination of characteristics described in Table 3.

TABLE 3

| Characteristic | ‘Mini Mels’ | ‘Mega Mels’ |
|---|---|---|
| Flower size. | Smaller than ‘Mega Mels’. | Larger than ‘Mini Mels’. |
| Abundance of | More abundant. | Less abundant. |
| involucral bracts. | | |
| Attitude of | More upright. | More relaxed. |
| involucral bracts. | | |
| General colora- | Dark purple; conspicuously | Light purple; inconspic- |
| tion of the | centered with a lighter | uously cenetered with a |
| flower. | shade of yellow. | darker shade of yellow. |

That which is claimed is:

1. A new and distinct cultivar of Stokesia plant named ‘Mini Mels’, substantially as described and illustrated herein.
